(19) United States
(12) Patent Application Publication
(10) Pub. No.: US 2024/0091059 A1
(43) Pub. Date: Mar. 21, 2024

(54) EYEDROP ADMINISTRATIVE DEVICE

(71) Applicant: PREEMIER, LLC, Southfield, MI (US)

(72) Inventors: Tammy Movsas, Southfield, MI (US); John Farris, Grand Rapids, MI (US); Cory Wyn, Grand Rapids, MI (US); Jenna Doyle, Grand Rapids, MI (US); Patrick Schwab, Grand Rapids, MI (US)

(21) Appl. No.: 18/468,868

(22) Filed: Sep. 18, 2023

Related U.S. Application Data
(60) Provisional application No. 63/411,677, filed on Sep. 30, 2022.
(60) Provisional application No. 63/407,804, filed on Sep. 19, 2022.

(51) Int. Cl. A61F 9/00

(52) U.S. Cl. CPC A61F 9/0026

(57) ABSTRACT

There is provided an eyedrop administrative device having a main device body, an eyedrop vessel for receiving and maintaining an eyedrop, a trajectory mechanism for expelling the eyedrop from the eyedrop vessel in an arced trajectory, thereby administering the eyedrop. Also provided is an eyelid lowering mechanism which is integrated into the eyedrop administrative device. Also provided is a method of administering an eyedrop by creating an eye pocket in the eye and expelling the eyedrop from the device in an arced trajectory from the device into the lower eyelid, also known as the eye pocket.

### CROSS-REFERENCE TO RELATED APPLICATIONS

This application claims priority to U.S. Provisional Patent Application No. 63/407,804, filed on Sep. 19, 2022, and U.S. Provisional Patent Application No. 63/411,677, filed Sep. 30, 2022, the entire contents of these applications are incorporated by reference.

### FIELD

The present technology relates to eyedrops. More specifically, the present technology relates to devices to administer eyedrops to an individual.

### INTRODUCTION

This section provides background information related to the present disclosure which is not necessarily prior art.

Eyedrop medicaments are used for a variety of eye conditions, eye diseases and eye disorders. Eyedrop users are challenged with the task of putting in eyedrops, a task that takes dexterity. This task can be especially challenging for individuals who are older, have co-morbidities such as tremors and arthritis, and/or have compromised eyesight and depth perception. This can be further complicated by the need to administer the eyedrops daily or even multiple times a day.

The task of administering the eyedrops is also complicated by the multiple requirements that must be performed for an eyedrop to be correctly administered. These requirements include the following: 1. Proper bottle alignment, which may require the line of eyesight to be blocked; 2. Opening of the lower eyelid with the hand that is not holding onto the eyedrop bottle for receipt of the eyedrop (i.e., pulling the lower eyelid down, thereby creating a pocket for receiving the eyedrop). Thus, eyedrop administration requires use of both hands. 3. Bending of the neck and tilting the head back so that gravity can be used to deposit the eyedrop into the eye 4. Squeezing the eyedrop bottle with just the right amount of pressure to discharge a single drop. 5. Making sure that the tip of the eyedrop bottle does not touch the eye so that contamination of the eyedrop bottle does not occur. This is made more onerous because eyedrops must often be administered in both eyes.

Many existing eyedrop aids on the market focus on proper placement of the bottle relative to the eye. Most of these devices require the head and neck to be tilted back. They function similarly to a normal eyedrop bottle in that once the eye bottle tip is aligned with the eye, gravity is used to pull the eyedrop down into the eye. However, tilting back of the head may cause dizziness and it is also particularly difficult for arthritic patients who experience pain when they tilt their necks. Some eyedrop aids have been developed that do not require the user to tilt their head back. One such delivery method instead attempted to pour the eyedrop into the eye.

While these attempts may overcome some limitations of eyedrop administration, they do not overcome all the problems described above. In addition, many eyedrop aids are designed for the eyedrop to be delivered to the center of an open eye (such as to the cornea). Eyedrops that hit the cornea often bounce off and run down the cheek, allowing only small fraction of the eyedrop to be absorbed into the eye. Instead, the American Academy of Ophthalmology and the National Eye Institute (which is part of the National Institute of Health), recommend that eyedrops are placed in the “eye pocket” which is formed when the lower eyelid is gently pulled down. The eye pocket provides sufficient volume to allow the eyedrop to reside there until absorbed by the eye.

### SUMMARY

Accordingly, there is a need to develop an eyedrop administration device for accurately and easily administering eyedrops without tilting the head or neck and that does not require great dexterity to use. Moreover, the device should deliver the eyedrops to the recommended landing place, namely, the eye pocket.

In concordance with the instant disclosure, an eyedrop administrative device, has been invented with the combination of the following features:
1. Trajectory Mechanism: The device of the present disclosure, when the eyedrop which has been loaded into the device is released, expel the eyedrop in an arced trajectory path, which results with the placement of the eyedrop within an eyelid pocket. The launch of the eyedrop does not depend on gravity, so it can be initially launched from below the line of sight, which minimizes patient/user anxiety.
2. Positioning Component: The device includes a positioning component, such as a contoured eyecup, which allows the user to accurately position the device on the face in a standardized way, regardless of facial anatomy. Moreover, the positioning of the device does not require the use of vision. For example, the user does not need to look into the mirror to position the device.
3. Natural Head Positioning: The device of the present disclosure allows the user to maintain a normal vertical head stance while applying eyedrops, eliminating the dizziness and discomfort caused by the usual requirement to tilt the head backward.
4. Handle with Cheek Bumper: The device's handle mechanism can include a cheek bumper. As the user activates the handle mechanism, the cheek bumper moves vertically downward, slightly pulling down the lower eyelid. This creates an “eye pocket” which serves as the ideal target for the incoming eyedrop.

The present technology includes articles of manufacture, systems, and processes that relate to a device for enabling the administration of an eyedrop to a user's eye. The device allows a user to administer eyedrops accurately using only one hand.

The device enables an individual to administer eyedrops easily and effectively to one's own eye. Alternatively, one can use the device to administer eyedrops to another's eye, which may be a person's eye or an animal's eye. The device is designed to accept at least one eyedrop from any sized eyedrop bottle. In the current embodiment, it accepts one eyedrop at a time. However, in an alternative embodiment, the device may accept more than one eyedrop at a time. The device includes the components for a trajectory mechanism for administering the eyedrop, a main device body for providing exterior support, an eyedrop vessel for accepting the eye drop(s) from the original eyedrop bottle, a positioning component for placement of the device on the user's face, and handle(s) with integrated cheek pad for lowering the bottom eyelid to form the eye pocket.

The trajectory mechanism is defined as a mechanism for causing an eyedrop to be expelled along a pre-determined arced trajectory to land in the eye pocket. The trajectory mechanism can be contained within the main device body or affixed to it.

The trajectory mechanism can be a pressurizing mechanism, which can draw in and then expel the eyedrop along a pre-determined arced trajectory. Alternatively, the trajectory mechanism can be a spring-loaded mechanism, wherein a cocked spring lever can be released to propel an eyedrop along a pre-determined arced trajectory.

There is provided an embodiment of the eyedrop administrative device having a device body, an eyedrop vessel (in the form of a removable tip), a trajectory mechanism within the device body for expelling the eyedrop from the tip in an arced trajectory, thereby administering the eyedrop. Also, provided is a method for administering an eyedrop via a trajectory mechanism for delivery into the eye pocket.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of an embodiment of an eyedrop administrative device of the present disclosure, including an integrated eyelid lowering mechanism.

FIG. 2 is an exploded view of the eyedrop administrative device of the present disclosure, including an integrated eyelid lowering mechanism.

FIG. 3 is a rear exploded view of the eyedrop administrative device of the present disclosure, including an integrated eyelid lowering mechanism.

FIG. 4 is a top perspective exterior view of the eyedrop administrative device of the present disclosure, including an integrated eyelid lowering mechanism.

FIG. 5 is a side exterior view of the eyedrop administrative device of the present disclosure, including an integrated eyelid lowering mechanism.

FIG. 6 is a bottom perspective view of an embodiment of the handle mechanism of the present disclosure.

FIGS. 7A-E are schematic drawings which depict the mechanism of the tip when it is connected to the pressurizing mechanism embodiment.

FIGS. 8A and 8B are schematic views of two different embodiments of an air pressurizing mechanism of the present disclosure.

FIGS. 9A and 9B shows the eyelid lowering mechanism of the present disclosure in use.

FIGS. 10A-10C are schematics of a spring-loaded mechanism, as the trajectory mechanism, for use in the device of the present disclosure. In this schematic, the eyedrop vessel is integrated into the mechanism.

FIG. 11 is a top perspective exterior view of another embodiment of the device of the present disclosure including a spring-loaded mechanism. In this figure, the spring-loaded mechanism is cocked and ready for eyedrop administration.

FIG. 12 is a partial side exterior view of the spring-loaded embodiment of the eyedrop administrative device after the eyedrop has been expelled.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below,” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves upon existing eyedrop aids. Generally, the present invention provides an eyedrop administrative device including a device body, an eyedrop vessel for receiving and maintaining at least one eyedrop, and a trajectory mechanism for expelling the eyedrop from the eyedrop vessel in an arced trajectory. The device can move the eyedrop along a trajectory from the eyedrop vessel into the eye. The device can be electronically or manually powered. The eyedrop administrative device can further include an eyelid lowering mechanism. Alternatively, the eyelid lowering mechanism can be separate from the eyedrop administrative device and be designed to function independently.

More specifically, the present technology provides an eyedrop administrative device, which facilitates the flow of eyedrops into the lower eyelid of the eye (“eye pocket”). Eyedrops are easily transferred, one drop at a time, from any eyedrop bottle, regardless of the shape and/or size of the original eyedrop bottle, into the eyedrop vessel of the device. When loading the eyedrops into the device, the tip of the original eyedrop bottle does not touch the device so there is no contamination of the eyedrop bottle tip. The present technology not only dispenses the medicine from the device into the eye but also assists the user in opening and holding open the lower eyelid of the eye for receipt of the eyedrop. Because of this, the eyedrop can be delivered from the device into the eye with the use of just one hand. In other words, the user's second hand is not needed to hold open the eye. When the lower eyelid is in a lowered position, an eye pocket is created, which is the recommended target for eyedrop delivery since the eyelid pocket can hold the drop's volume until it is absorbed by the eye.

Since the device does not depend upon gravity to launch the eyedrop, the eyedrop administrative device can launch the eyedrop from below the line of sight. This serves two important functions, first it substantially reduces “eyedrop anxiety” since the user cannot see the approaching eyedrop. Second, it lowers the risk of any type of injury from the device falling or impacting the user's eye since the device can be held vertically against the face of the user. Therefore, the safety of the device and the user's experience can be enhanced. When the eyedrop administrative device is used, the eyedrops follow an upward and then downward trajectory and ultimately gently lands in the “eye pocket” (i.e., an opening formed when the lower eyelid is lowered).

The device enables an individual to administer eyedrops easily to one's own eye. Alternatively, one can use the device to administer eyedrops to another's eye, which may be a person's eye or an animal's eye. The person or animal may be of any age.

In the present disclosure, the eyedrop administrative device can also include an eyelid lowering mechanism. Alternatively, the eyelid lowering mechanism can be used without the main device body.

The eyelid lowering mechanism can include a positioning component, a cheek bumper, and a main handle body. The positioning component can help position the device in a standard way relative to the eye and the lower eyelid. The cheek bumper rests on the cheek of the user and when the eyelid lowering mechanism can be activated, the cheek bumper can move vertically downward, gently pulling down the lower eyelid, creating an eye pocket. The main handle body can be designed to engage with an eyedrop administrative device or can function as an independent device. F

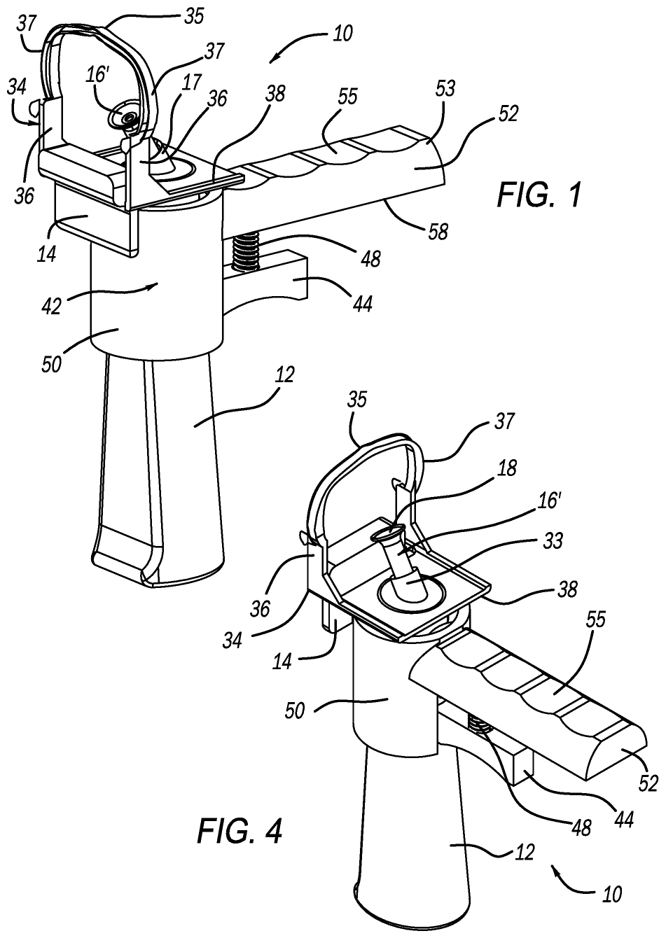

Referring generally to an embodiment of the device of the present disclosure as shown in FIG. 1, the eyedrop administrative device 10 can include a main device body 12, an eyedrop vessel 16, and a trajectory mechanism 24 (not shown in FIG. 1) integrated with the main device body 12. Another embodiment, the trajectory mechanism can be attached to the device body, not integrated within the main device body 12.

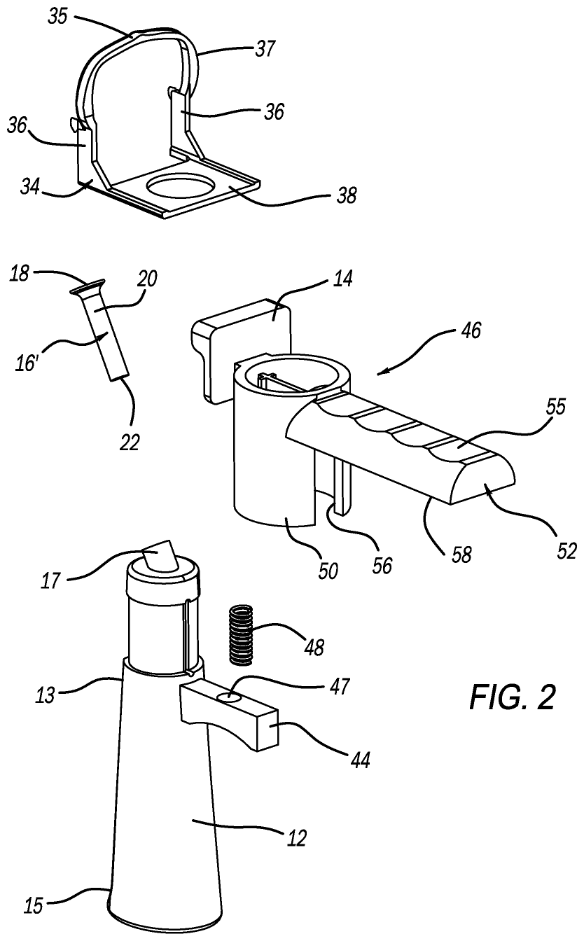

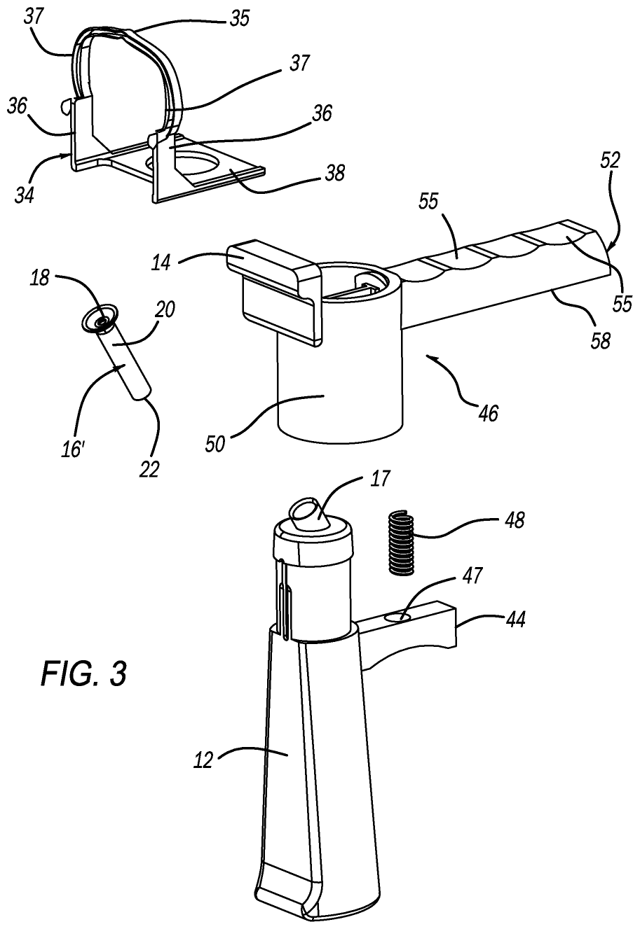

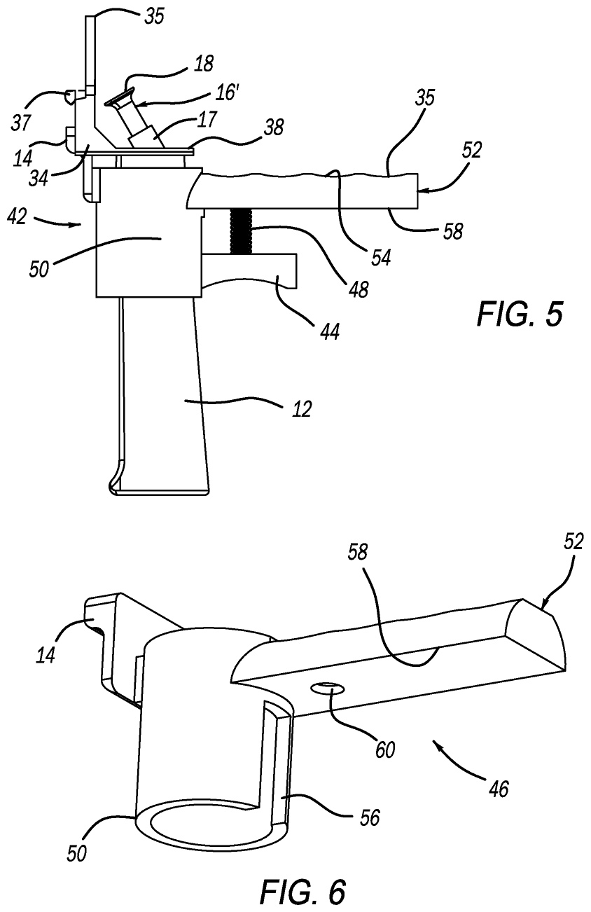

The main device body 12 can be formed of a resilient material that can be non-reactive and biocompatible such that if the housing is in contact with the face of the user, minimal or no adverse reaction can occur. The main device body 12 can be a solid piece of material or can be a hollow shell that can encompass other components of the eyedrop administrative device 10. Some non-limiting examples include a combination of rubber, metals and plastics, examples of which are well known to those of skill in the art. This material can be a rigid or hard material to provide additional strength to the device. As shown in the FIGS. 1-6, the main device body 12 has a top 13 and a bottom 15. The bottom 15 can be formed with a flat base to ensure device stability. Alternatively, the bottom 15 can include means for engaging a recharging base (not shown).

The eyedrop vessel 16 can be disposable, removable, and/or re-usable. The eyedrop vessel 16 can be formed of a material that can either be sterilized, sanitized, or disposed. The eyedrop vessel 16 can be flexible or rigid. Additionally, the material can be formed of a material that will not react with the eyedrops, preferably with hydrophobic properties. Some non-limiting examples include, but are not limited to, plastics and biocompatible polymers. Other materials can also be used without departing from the spirit of the present disclosure.

The eyedrop vessel 16 can be capable of receiving and retaining at least one eyedrop 31 from an eyedrop bottle until it is dispensed. In an embodiment, the eyedrop vessel 16 can hold the volume of one drop of eye medicine (approximately 15 to 100 microliters). This allows the user to dispense the correct dosage of medicine (i.e., one drop of eye medicine) into the eyedrop vessel 16 prior to dispensing the medicine into the eye. This prevents the user from accidentally over-dispensing eyedrop medicine (such as by over squeezing the eyedrop bottle) while administering the medicine. When multiple eyedrops are required, the user may administer one eye drop at a time. Alternatively, the eyedrop vessel 16 can be sized sufficiently large so as to accommodate the volume of two or more eyedrops at a time.

The eyedrop vessel 16 can be of any shape capable of maintaining an eyedrop 31. Examples of such shapes include, but are not limited to, rectangular, semi-circular, cylindrical receptacles. The eyedrop vessel 16 can be built into the administrative device 10, attached to the administrative device 10, or removable.

In an embodiment of the present disclosure, the eyedrop vessel 16 can be in the shape of a specialized tip 16′. The tip 16′ can be fixed in place, removable, and/or disposable. Thus, enabling the tip 16′ to be either thrown away or cleaned after use, thus, reducing or eliminating the chance of contamination, thereby reducing chance of infection. Additionally, the tip 16′ can include an eyedrop receptacle 18, an eyedrop maintaining core 20, and an air evacuating tip 22. The eyedrop receptacle 18 can be designed to both accept the eyedrop, without affecting the sterility of the eyedrop, and can also funnel the eyedrop into the eyedrop maintaining core 20. The eyedrop receptacle 18 can be formed as any shape that effectuates the disclosed parameters. Some non-limiting examples of such shapes include, but are not limited to, a V-shape and a U-shape. The eyedrop maintaining core 20 can keep the eyedrop in a temporary location prior to administration. The purpose of the eyedrop maintaining core 20 is to prevent contamination and to allow for more effective administration of the eyedrop. Additionally, the tip 16′ can be formed as a single unit with the device, can be a removable piece, and/or can be a disposable piece. The tip 16′ can formed of a material that can be sterilized or otherwise cleaned. For its proper functioning, the tip 16′ can be open on both ends.

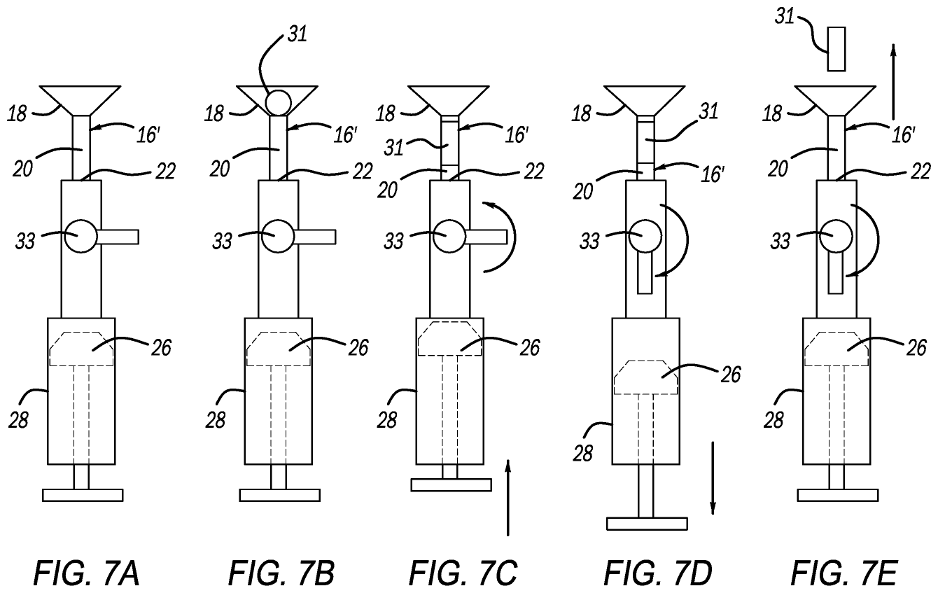

FIGS. 7A-7E are schematics showing how the eyedrop 31 can be retained within the tip 16′, even though the tip can be open on both ends. The eyedrop 31 does not fall below the eyedrop maintaining core 20 at any time because of both the surface tension of the eyedrop 31 and the column of air trapped below the eyedrop 31 in the air evacuating tip 22.

In an embodiment of the present disclosure, the tip 16′ can be inserted into the administrative device 10 at a tip receptacle 17. The tip receptacle 17 can maintain the 16′ in a fixed angled position within the administrative device 10, such that the tip 16′ can be angled toward the eye for the administration of the eyedrop 31.

In another embodiment of the present disclosure, the tip 16′ can be inserted into the administrative device 10 at a tip receptacle 17 in a vertical configuration. This makes it easy to both insert the tip 16′ into the administrative device 10 as well as to load an eyedrop 31 into the tip 16′. After the eyedrop 31 is loaded, the tip receptacle 17 can be move into a tilted position, thereby angling the tip 16′ towards the eye, to optimize the angle at which the drop can be expelled out of the tip 16′.

The trajectory mechanism 24 can be any mechanism capable of expelling the eyedrop 31 from the eyedrop vessel 16, via a trajectory, into the eye pocket 19. The eyedrop vessel 16, from which the eyedrop 31 can be expelled, can be located below the eye; this keeps the eyedrop 31, when it is launched, out of the patient's sight. To administer the eyedrop 31 to the user's eye, the administrative device 10 may move the air behind the eyedrop 31, which, in turn, moves the eyedrop 31 and gives it the desired velocity and acceleration. However, the trajectory mechanism 24 can be any projectile motion capable device. Examples of such mechanisms are well known to those of skill in the art, and can include, but are not limited to, air pressure (puff of air method) and pressurization devices and spring-loaded mechanisms. The movement of the eyedrop 31 can be in a predetermined trajectory, such that upon activation of the device the eyedrop 31 can be expelled from the device in a trajectory and then flows into the eye. Some non-limiting examples include bellows, syringe-like mechanism, or an air compression plunger. Other mechanisms to move an eyedrop 31 in a predetermined trajectory are known to those of skill in the art and can also be used without departing from the spirit of the present invention.

More specifically, the trajectory of the drop of medication can be dependent on the velocity. For example, when 1.22 m/s is the maximum exit velocity of the drop from the eyedrop vessel 16 and that the drop hits the eye at the apogee of its parabolic motion. Alpha is the angle of the eye drop dispenser relative to the horizon. At the apogee, the velocity of the drop in the vertical direction can be zero and in the velocity in the horizontal direction a function of the angle alpha. If the eyedrop vessel 16 can be positioned at an angle of 45° from the horizon, then the drop would hit the eye pocket 19 with a velocity of 0.86 m/s. The apogee of the path would be 37.8 mm above the outlet of the eyedrop vessel 16 and 100 mm to in front of the outlet of the tip. The horizontal and vertical distances the drop of medicine travels can be controlled by the magnitude of the angle alpha and the pressure of the air used to launch the drop. Preliminary testing has shown that the required distances can be achieved with compressed air at a pressure below 110 Kpa or 16 psi.

In an embodiment of the present disclosure, the trajectory mechanism 24 can be an air pressurizing mechanism such as a bellows or syringe of some kind, can be used to create the pressurized air. Other mechanisms that allow air into them when they are expanded and expel air out of them when they are condensed can also be used without departing from the spirit of the present invention.

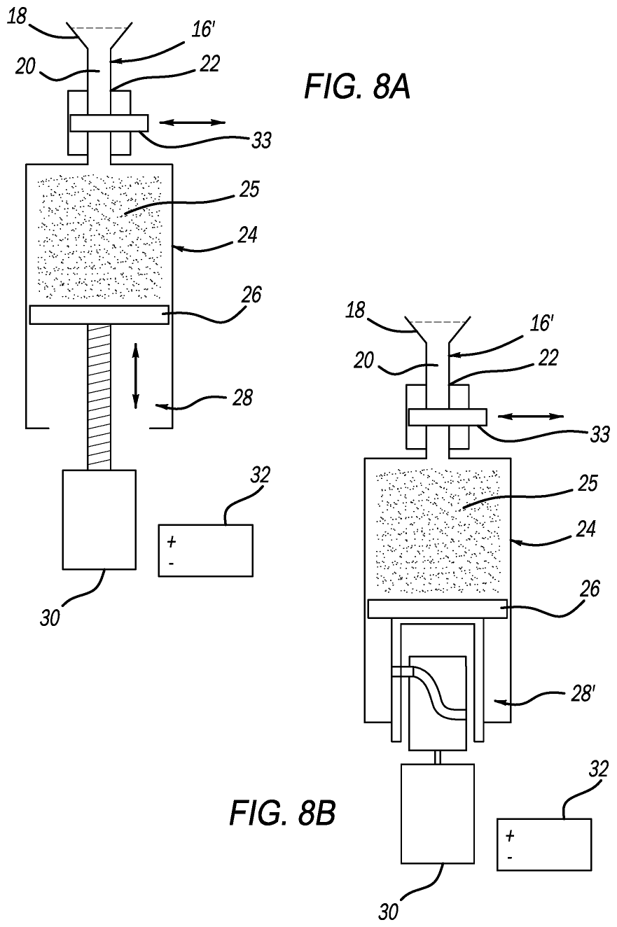

In an embodiment, the trajectory mechanism 24 can be an air pressurizing mechanism, which is shown in the FIGS. 7 and 8, as an air reservoir 25 and a plunger 26. In order for the air pressurizing mechanism 24 to function, it can be connected to a motion activating mechanism 28. The motion activating mechanism 28 can be used to turn the rotational motion of the motor into linear motion to move the plunger 26. Other mechanisms for providing this motion can be used in the device, examples of which are known by people of skill in the art, without departing from the spirit of the present invention.

The motion activating mechanism 28, can be any mechanism that can provide a sufficient force to first suck the drop further down into the tip and then cause deployment of an air pressurizing mechanism which can be sufficient to move the eyedrop 31 from the eyedrop vessel 16 to the eye pocket 19. To effectuate this, the administrative device 10 can further include an electronic valve 33 to regulate the flow or air into and out of the administrative device 10. Examples of valves 33 include, but are not limited to, a solenoid or a mechanically actuated valve. The electronic valve 33 which can be activated to open, thus allowing the system to draw external air into the system when the plunger 26 moves down as shown in FIG. 7C. The motor 30 can activate the lead screw 28 in a preset direction, for example, counterclockwise. This can move the plunger 26 down, drawing the eyedrop 31 into the eyedrop maintaining core 20, by creating negative pressure. The electronic valve 33 can close. The user can position the administrative device 10 in front of the eye and activate the expulsion of the eyedrop 31. Upon activation of the expulsion, the motor 30 can rotate the lead screw 28 in a direction opposite to the previous motion, clockwise. This can move the plunger 26 up, building positive pressure in the air reservoir 25 as shown in FIG. 7D.

After the plunger 26 has moved a predetermined distance up and sufficient pressure has been achieved the electronic valve 33 can open. The positive pressure of the air accelerates the eyedrop 31 out of the tip. After the valve 33 has opened, the motor 30 can continue to rotate lead screw 28 in order to continue the flow of air exiting the tip. The plunger 26 continues moving up after the valve 33 has opened to continue to push air out of the device. This, in turn, can help reduce volume of the eyedrop 31 left in the tip. In other words, positive pressure can be used to accelerate the eyedrop 31 out of the tip even after the eyedrop 31 has left the tip and the motion activating mechanism continues to allow the flow of air exiting the tip 16′. This can be a novel methodology for reducing residuals in the system. The motor 30 can return the plunger 26 to the starting position by rotating the lead screw 28 in the opposite direction. The closing the electronic valve 33 can close and the system can be ready for another cycle.

As shown in the FIGS. 8, the motion activating mechanism 28 can employ the use of a motor 30. The eyedrop 31 flows up against gravity, in a trajectory-like path, resembling the path of water from a drinking fountain. Some non-limiting examples of such motion activating mechanisms 28 include, but are not limited to, a linear actuator, a motor, a motor with a regular cam, a motor with a lead screw, and a motor with a barrel cam. Someone skilled in the art can determine the best type of motor for the intended purpose. Non-limited examples of motors include, but are not limited to, a small DC motor driven by battery or a stepper motor. In an embodiment, the administrative device 10 can be powered by a small electric motor 30, the motor 30 can include a battery 32, including, but not limited to rechargeable batteries. While specific mechanisms are disclosed herein, other mechanisms that are capable of providing the administration of the eyedrop 31 as disclosed herein can also be used without departing from the spirit of the present invention.

A schematic of an embodiment of the present disclosure is shown in FIG. 7. The schematic depicts how the tip 16′ can accept the eyedrop 31 and subsequently expels the eyedrop 31 from the administrative device 10. FIG. 7A identifies the parts of the administrative device 10. The motor 30, battery 32 and motion activating mechanism 28 have been left out for clarity. FIG. 7B shows the eyedrop 31 in the receptacle 18 of the tip 16′. The valve 33 can be closed to prevent the eyedrop 31 from falling into the air evacuating tip 22. Next, the valve 33 can be opened, and the plunger 26 moves down to suck the eyedrop 31 into the drop maintaining core 20 as shown in FIG. 7C. In FIG. 7D, the valve 33 can be shut, and the plunger 26 can be moved upward to build up pressurized air behind the valve 33. Finally, the valve 33 can be opened and the eyedrop 31 can be propelled by the pressurized air out of the administrative device 10 and into the user's eye pocket 19. The plunger 26 continues to move upward until the drop has left the tip 16′. Then plunger can be returned to the start position and valve 33 can close as shown in FIG. 7E. The system can be ready to accept another drop. The following conditions can also be utilized for reducing the volume of the residual left in the tube: Volume of pressurized air=20 mL (Higher is better); Speed of Plunger=750 mm/min (Faster is better); Tube size=1.59 mm in diameter. (Smaller is better).

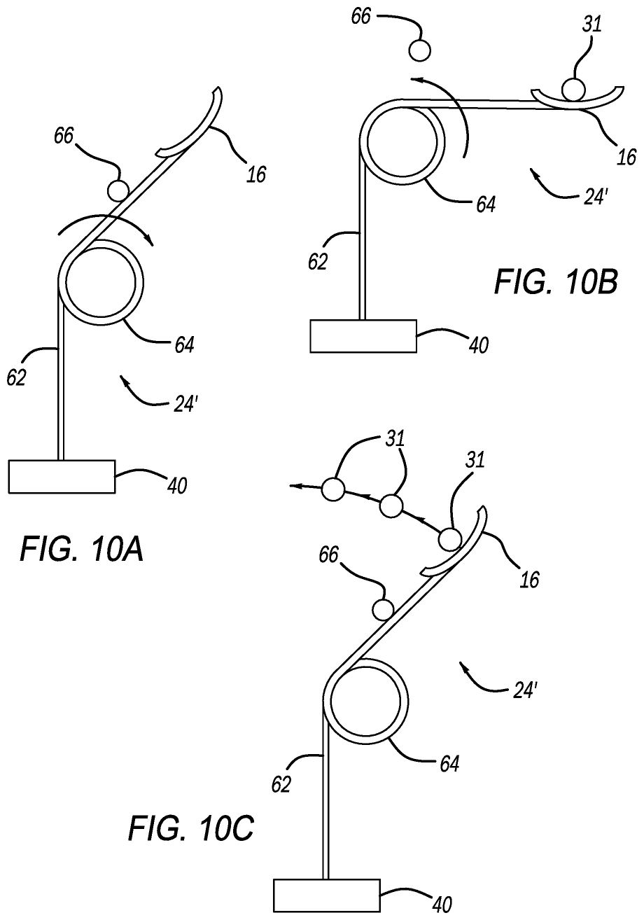

In another embodiment, the trajectory mechanism 24′ can be a spring-loaded mechanism, as shown in FIG. 10. The spring-loaded mechanism 24′ can be positioned in cocked or loaded position, such that when released, spring loaded mechanism 24′ can propel an eyedrop 31 along a pre-determined arced trajectory into the eye pocket 19. Examples of such spring-loaded mechanisms 24′ include, but are not limited to, torsional, compression disc, flat or leaf springs, amongst other types of springs. Examples of such spring-loaded mechanisms 24′ are known to those of skill in the art. The spring-loaded mechanism 24′ can have include a specially shaped eyedrop vessel 16 attached to it or integrated within it. The eyedrop vessel 16 can have shapes that include, but are not limited to, semi-circular, rectangular, square, cylindrical receptacles, and other shapes known to those of skill in the art.

More specifically, as shown in FIG. 10, the trajectory mechanism 24′ can include an arm 62, a torsional spring 64, a fixed stop 66 for preventing the arm 62 from proceeding past a set point, and an eyedrop vessel 16. In use, the arm 62 can be pulled backward, then an eyedrop 31 can be placed on an eyedrop vessel 16 of the arm 62. Then the arm 62 can be released to the fixed stop 66, whereby the eyedrop 31 can be expelled from the eyedrop vessel 16, along a pre-calculated trajectory 68 into the eye pocket 19. The trajectory of the eyedrop 31 depends on the force and torque of the torsional spring 64 and angle of release, which can be determined by one of skill in the art to effectuate the intended results.

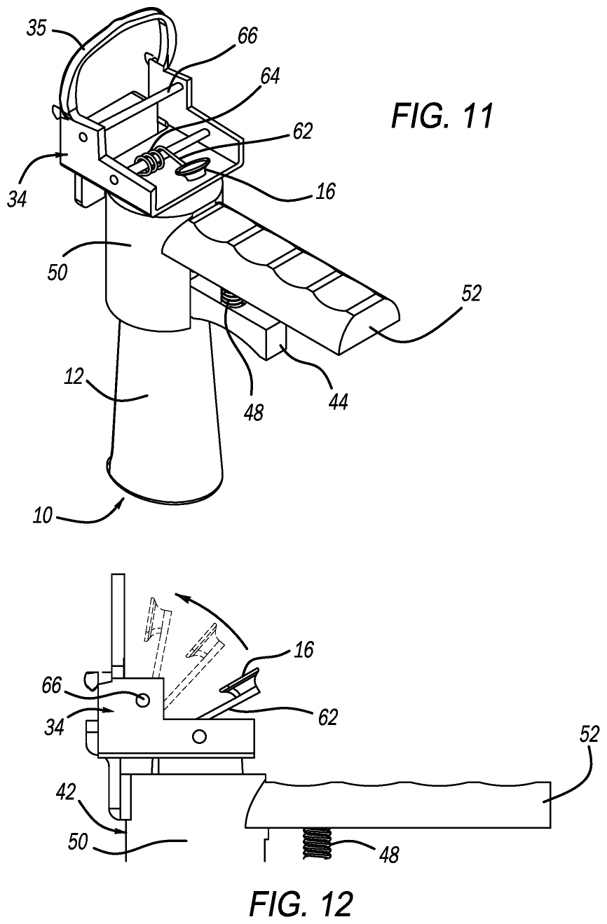

In one embodiment, shown in FIG. 11 and FIG. 12, the spring-loaded mechanism 24′ can be affixed to the main device body at a position approximately below the eyecup. When the eyedrop 31 can be launched from this positioning, the eyedrop 31 can approximately land in the center of the eye pocket 19. Alternatively, the spring-loaded mechanism 24′ can be attached to the main device body 12 via a lateral attachment (not shown). In this alternative embodiment, the eyedrop 31 can be launched from a position lateral to the center of the eye and therefore, lands along the length of the eye pocket 19, which may not necessarily be right under the center of the eye. The lateral positioning mechanism can be used to increase the size of the targeted landing area within the eye pocket 19.

As seen in FIG. 1, the administrative device 10 can also include a positioning component 34. The positioning component 34 can be a contoured eyecup. The positioning component 34 helps the eyedrop 31 user to position the administrative device 10 into a standardized position against the face. The positioning component 34 can be attached to the top 13 of the main device body 12. The positioning component 34 can be formed of a resilient material that can be non-reactive and biocompatible such that if the administrative device 10 is in contact with the face of the user, minimal or no adverse reaction can occur. Some non-limiting examples include a combination of rubber, metals and plastics, examples of which are well known to those of skill in the art.

As seen in FIGS. 1-5, in an embodiment of the present disclosure, the positioning component 34 can have a generally oval shaped upper portion 35 that can be sized to rest on the eye socket of a user to assist in creating the proper alignment of the administrative device 10. The oval shaped upper portion 35 can extend down to arms 36 that can affix the upper portion 35 to a base unit 38.

The entire positioning component 34 can be made as a single unit or can be individual components that are affixed to one another. The arms 36 can also include soft flanges 37 to provide a more comfortable fit for the positioning component 34. The base unit 38 can include a flat piece of material, with optional raised edges. The center of the base unit 38 can include an aperture through which the tip 16 of the device extends. The positioning component 34 can be contoured to fit the superior eye orbit. However, unlike eyecups used to position other eyedrop aids, this positioning component 34 can be open at the bottom. This can allow the eyedrop 31 to have direct access to its eye pocket 19 without any physical interference.

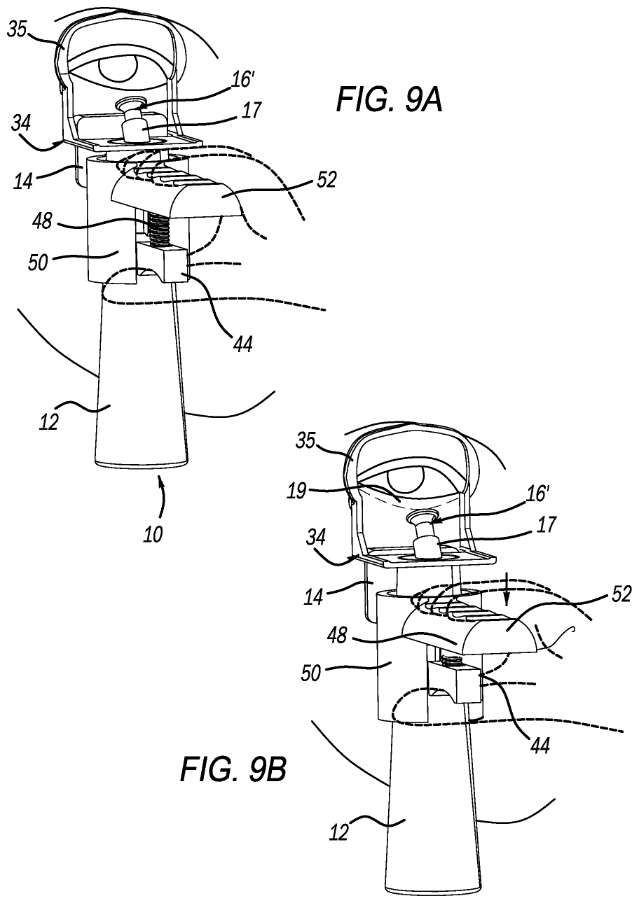

As shown in FIG. 9, in use, the administrative device 10 can be grasped by the handle 52 and thumb grip 44, and the positioning component 34 can be placed over the eye to allow for positioning of the administrative device 10 against the face. The positioning component 34 can help to properly place the eyelid lowering mechanism 40. The positioning component 34 does not require the use of vision to place it. In other words, the patient does not need to look into the mirror to properly place the eyelid lowering mechanism 40 against the face. When the administrative device 10 has been positioned, the main device body 12 can be vertically upright, to some extent, such that it does not require the tilting of the head or neck to operate. The main device body 12 can be positioned and operated with just one hand.

In another embodiment of the present disclosure, the positioning component 34 can resemble the bridge and nose pads of an eyeglass frame which can be engineered by someone skilled in the art to function as a positioning device similar to how eyeglasses fit on an eyeglass wearer's face (not shown).

The present embodiment of the administrative device 10 also includes an eyelid lowering mechanism 40. The eyelid lowering mechanism 40 can be any mechanism that causes the cheek pad to be vertically lowered to create the eye pocket 19. The eyelid lowering mechanism 40 can be activated, either mechanically or electronically, activating a handle system 42. The handle system 42 can include a thumb grip 44, a handle mechanism 46, and an interconnecting mechanism 48 for connecting the thumb grip 44 and handle mechanism 46. The handle system 42 can be activated by squeezing together the handle mechanism 46 and the thumb grip 44. Finally, when the eyelid has been lowered to the proper position, the eyedrop 31 can be expelled from the main device body 12 to be administered to the eye. The eyedrop administration can occur either manually or can be automated.

The thumb grip 44 can be affixed to an exterior surface of the main device body 12. The thumb grip 44 can preferably be made of a similar material as that of the main device body 12. The thumb grip 44 can be generally rectangular or can be formed with a concave surface to allow for ease in use of the device. On the top surface 45 of the thumb grip 44 there can be a hole 47 for receiving the interconnecting mechanism 48.

The handle mechanism 46 can include a main handle body 50 and a handle 52 extending out from the main handle body 50. The main handle body 50 can be formed to engage with an outer surface of the main device body 12. The main handle body 50 can be shaped to slide over the main device body 12 such that it enables the main device body 12 to be retained within or otherwise engaged with the main handle body 50 of the handle mechanism 46. Additionally, the main handle body 50 can include an aperture 56 shaped to accommodate the thumb grip 44, such that the thumb grip does not impact or otherwise touch the main handle body 50 during operation.

Alternatively, the handle mechanism 46 can include a variety of handle types including, but not limited to, lever handles, pull handles, twist handles, clamp handles. Depending upon the handle type, the thumb grip 44 may be replaced with alternative parts performing the same function or rendered unnecessary. The handle mechanism 46 can interconnect with the main device body 12, in a variety of ways, including, but not limited to, can be integrated with the main device body, or can be attached to it. Such functional alternative can be considered to be within the spirit of the present disclosure.

Additionally, a cheek bumper 14 can be affixed to the exterior of the main handle body 50. The main handle body 50 can be formed as a single unit or as separate pieces that can be engaged or otherwise interconnected.

The cheek bumper 14 can be a padded material that can be affixed to the exterior of the main device body 12. The cheek bumper 14, in addition to the positioning component, 34, can be used to provide proper alignment of the administrative device 10 on the face of the user. The primary function of the cheek bumper 14 can be to open the lower eyelid creating an eye pocket 19 into which the eyedrop 31 can be placed (See FIG. 9B).

The cheek bumper 14 can be formed of a soft resilient pad. The cheek bumper 14 can be formed of any biocompatible, and preferably hypo-allergenic material, and optionally out of a rubber-like material, that can be attached to the exterior surface of the administrative device 10. The cheek bumper 14 can be designed to be placed against the face of the user (over the lower orbital rim). While the cheek bumper 14 can be being held against the user's face, the handle system 42 creates a downward direction. The downward motion gently pulls down the facial skin, thus lowering the lower eyelid. This in turn, creates an eye pocket 19 into which the eyedrop 31 can be administered.

In the present embodiment, the handle 52 extends out from the main handle body 50 and can be located above the aperture 56. The handle 52 can be preferably made of a similar material as that of the main device body 12. The handle 52 can be generally rectangular with curved sides shaped to accommodate a human hand. The top surface 54 can further include ridges 55 to accommodate the fingers of a human hand. The handle 52 can also include a non-slip material on the surface 54 to prevent slippage during use. On the bottom surface 58 of the handle 52 there can be a hole 60 for receiving the interconnecting mechanism 48.

The interconnecting mechanism 48 can be any mechanism known to those of skill in the art to enable a present compression and subsequent release of such compression to occur repeatedly. For example, the interconnecting mechanism 48 can be a spring having a present tension determined by one of skill in the art.

In an embodiment, the user, with one hand, gently squeezes together the handle 52 and the thumb grip 44, which in turn moves the cheek bumper 14 vertically downward, gently pulling down the lower eyelid. The coefficient of friction between the cheek bumper 14 and skin can be sufficient so as to move the skin with the cheek bumper 14, pulling the lower eyelid down and creating a pocket beneath the eye, an eye pocket 19, into which the eyedrop 31 can be administered. In alternative embodiments, the activation of the cheek bumper 14 to lower the eyelid can be facilitated by alternate mechanism known to those of skill in the art. Variations of the above eyelid lowering mechanism 40, including the handle mechanism 46, and the attachment of the handles 52 to the main handle body 50, which can be developed by one of skill in the art can also be used without departing from the spirit of the present disclosure.

Prior to use, the handle mechanism 46 can be in an open position, wherein the handle 52 and thumb grip 44 are not in close proximity. By compressing the interconnecting mechanism 48, the handle 52 and thumb grip 44 are brought closer together. When this occurs, the cheek bumper 14, which can be connected to the handle mechanism 46 can be physically pulled down. This lowers the lower eyelid forming the eye pocket 19. While the handle mechanism 46 moves, the eyedrop vessel 16 does not move. This ensures that the eyedrop 31 can be always launched from a fixed pre-determined position, which allows it to follow a pre-determined, pre-calculated trajectory.

In another embodiment, the eyelid lowering mechanism 40 can be built separately from the main device body 12 and can function as a solitary unit (not shown). The eyelid lowering mechanism 40 can include: an integrated handle system 42, a main device body 10, (which can be adapted to integrate with the main device body 12 of the eyedrop administrative device 10, a positioning component 34 which can be attached to the handle system 42, and a cheek bumper 14 can be attached to handles 52 of the handle system 42. Alternatively, the handles 52 can be integrated into the main device body 12 of an eyedrop administrative device 10 or can be affixed to the main device body 12.

In an alternate embodiment, a patient may utilize the eyelid lowering mechanism 40, as an independent device, to open his lower eyelid, which, in turn, allows a second person (such as a caregiver) to administer eyedrops to the eye pocket 19 of the patient. The method to utilize the eyelid lowering mechanism 40 as an independent device would be for a person to position the device with the positioning component and then compress the handles 52 of the eyelid lowering mechanism 40 to activate the eyelid opener to lower the eyelid.

The eyedrop administrative device 10, when in use, can be held with one hand in a position that is primarily upright. A front and side view of the administrative device 10 in this orientation can be seen in FIGS. 9A and 9B. Importantly, neither the positioning component 34 nor the eyedrop vessel 16 move when the eyelid lowering mechanism 40 can be activated. In other words, when the lower eyelid moves down to create the eye pocket 19, the eyedrop vessel 16 (from where the drop will be launched) remains in a fixed position. This ensures that the eyedrop 31, when it can be launched, will be expelled from a fixed pre-determined position so that it will follow the pre-calculated trajectory and land in the correct location.

Once in position, the user activates the administrative device 10, such as by the push of a button, and the eyedrop 31 can be projected into the eye pocket 19. Alternatively, when the cheek bumper 14 can be lowered to the correct position, it may automatically trigger the release of the eyedrop 31.

In an embodiment with a disposable eyedrop vessel 16, the eyedrop vessel 16 can be discarded after use. In an embodiment with charging base, the administrative device 10 should be placed in charger after use. In cases where patients need to administer more than one eyedrop 31 to the eye or need to administer eyedrops to the other eye, the above steps can be repeated. Variations of the above methodology which yield substantially similar results, should be considered to be within the same scope and spirit of the invention.

In another embodiment, the administrative device 10 can include a time-delay mechanism that creates a short delay prior to administration of the drop. In such an embodiment, the device can dispense the eyedrop 31 after a period of time which gives the user enough time to properly place the device. The time delay may range between 3 seconds and 2 minutes. In another embodiment, the expulsion of the eyedrop 31 can be triggered to occur nearly simultaneously with the creation of the eye pocket 19.

In another embodiment, the administrative device 10 can be engineered, with the use of a lateral positioning mechanism, such that the trajectory mechanism can be positioned lateral to the main device body using a lateral connection. Thus, the eyedrop 31 can be expelled laterally (i.e., from a position closer to the outer corner of the eye) towards the center of the eye pocket The positioning of the positioning component and eyelid lowering mechanism 40 would not change; only the positioning of the trajectory mechanism 24 changes. With this alternate positioning of the trajectory mechanism 24, the eyedrop 31 can enter the eye pocket 19 at a position along its length, and not necessarily at the center of the eye pocket 19. This may be advantageous because the eye pocket 19, (i.e., the targeted landing place) can be longer than it is deeper. Whereas it is about 14 mm in length (ranges from about 11.8 to 16.2), it is only 3-6 mm in depth. Therefore, when the eyedrop 31 can be launched towards the eye pocket 19 from a position that can be lateral to center of the eye, there can be a larger target area for the eyedrop landing place.

In another embodiment, the administrative device 10 can include a rechargeable battery that can be charged by having the administrative device 10 sit in a charging base. Alternatively, the administrative device 10 can contain a disposable battery, or the administrative device 10 can be activated by being plugged into an electric outlet. In use, the administrative device 10 starts in a base station on a flat surface. The base station tilts the device so that the tip can be easily accessible. The base station can also contain the electronics required to charge the battery in the device. The remaining steps are as described above.

In another embodiment, the administrative device 10 can include a bottle receiving and/or retaining mechanism for enabling the eyedrop bottle to be maintained within the device. This can allow the administrative device 10 to remove the eyedrop 31 from the eyedrop bottle on an as-needed basis without requiring any additional activity by the user.

The method of use of the administrative device 10 is generally as follows. First, an eyedrop 31 can be loaded into the eyedrop vessel 16. Then, the user grasps the administrative device 10 with one hand, with four fingers on the handle 52 and thumb on the thumb grip 44 and the administrative device 10 can be placed, and held in place, against the face of the user, in by placing the positioning component 34 over the eye and pressing it against the skin. The head can be held upright, with no need to tilt the head backwards. FIG. 9A shows a patient with the administrative device 10 in position for use. Next, the user activates the eyelid lowering mechanism 40 by closing the gap between the handle 52 and the thumb grip 44 such as by compressing the spring 48 between the handle 52 and thumb grip 44. This causes the cheek bumper 14 to pull the user's lower eyelid down slightly, thereby exposing the eye pocket 19 as shown in FIG. 9B. Next, the trajectory mechanism 24 of the administrative device 10 can be activated which causes the eyedrop 31 to be expelled up and out of the administrative device 10 and into the lower eyelid pocket. The eyedrop 31 can be expelled from below the eye, (i.e., below the line of sight of the user). As soon as an eyedrop 31 has landed in the eye, the patient can remove administrative device 10 from their face. All steps can be done with only one hand, do not require a mirror, and do not require tilting the head. If more than one eyedrop 31 is required, the patient can repeat the steps above.

Example embodiments are provided so that this disclosure will be thorough, and will fully convey the scope to those who are skilled in the art. All calculations, described within this patent application, (such as for desired acceleration and velocity) may vary depending on a variety of factors. Non-limiting examples of these factors are tubing length, tubing diameter, tubing material, type and strength of motor, and the user's individual anatomy. However, a person of skill in the art can determine the specifics using routine experimentation to determine the exact lengths, angles, etc. Any of the ranges of variables (contained within this application) may fall outside the indicated ranges when all factors are considered. Therefore, after some of the variables are finalized, dimensions and calculations can be performed by someone skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. An eyedrop administrative device comprising:
a main device body;
an eyedrop vessel for receiving and maintaining at least one eyedrop, the eyedrop vessel engaged with a trajectory mechanism; and
a trajectory mechanism, engaged with the main device body, for expelling the eyedrop from the eyedrop vessel in an arced trajectory.

2. The device according to claim 1, wherein the trajectory mechanism is selected from the group consisting of a pressurizing mechanism, a spring-driven mechanism, a motorized pressurizing mechanism, and a motorized spring-driven mechanism.

3. The device according to claim 2, wherein the trajectory mechanism further includes a lateral positioning mechanism for expelling the eyedrop laterally.

4. The device according to claim 1, further including a positioning component attached to the main device body.

5. The device according to claim 4, wherein the positioning component is an eyecup.

6. The device according to claim 1, wherein the eyedrop vessel is selected from the group consisting of a tip having an open receiving end and an open pressurizing end, semi-circular, rectangular, square, and cylindrical shaped receptacles.

7. The device according to claim 1, further including a cheek bumper.

8. The device according to claim 1, further including an eyelid lowering mechanism comprising:
a main handle body including a positioning component;
a handle mechanism engaged with the main handle body; and
a cheek bumper engaged with the handle mechanism;
wherein the eyelid lowering mechanism opens a lower eyelid upon engagement of the handle mechanism.

9. The device according to claim 8, wherein the handle mechanism includes:
a thumb grip attached to the main handle body;
a handle interconnected with the main handle body and matingly engaged with the thumb grip; and
an interconnecting mechanism for interconnecting the thumb grip and handle.

10. An eyelid lowering mechanism comprising:
a main handle body including a positioning component;
a handle mechanism engaged with the main handle body; and
a cheek bumper attached to the handle mechanism; and
wherein the eyelid lowering mechanism opens a lower eyelid upon engagement of the handle mechanism.

11. The eyelid lowering mechanism according to claim 10, wherein the eyelid lowering mechanism is engaged with and an eyedrop administrative device, the eyedrop administrative device comprising:
a main device body;
an eyedrop vessel for receiving and maintaining at least one eyedrop, the eyedrop vessel engaged with a trajectory mechanism; and
a trajectory mechanism, engaged with the main device body, for expelling the eyedrop from the eyedrop vessel in an arced trajectory.

12. The eyelid lowering mechanism according to claim 10, wherein the handle mechanism includes:
a thumb grip engaged with the main handle body;
a handle affixed to the main handle body and matingly engaged with the thumb grip; and
an interconnecting mechanism for interconnecting the thumb grip and the handle.

13. A method of administering an eyedrop using the eyedrop administrative device of claim 1, comprising the steps of:
loading the eyedrop vessel of the device with an eyedrop;
positioning the device against a user's face; and
expelling the eyedrop from the device in an arced trajectory from the device.

14. A method of administering an eyedrop using the device of claim 8, comprising the steps of:
loading the eyedrop vessel of the device with an eyedrop;
aligning the device on a face with the positioning component;
creating an eye pocket in an eye; and
expelling the eyedrop from the device in an arced trajectory into the eye pocket.

15. The method according to claim 14, wherein the method further includes administering an eyedrop using one hand.

16. The method according to claim 14, wherein the method further includes administering an eyedrop without tilting the head or neck.

17. The method according to claim 14, wherein the method further includes administering an eyedrop without using a mirror.

18. The method according to claim 14, wherein the aligning step includes aligning the device with the eyedrop vessel positioned below a line of sight.

19. A method for creating an eye pocket using the eyelid lowering mechanism of claim 10, comprising the steps of:
aligning the eyelid lowering mechanism using the positioning component; and
activating the eyelid lowering mechanism with the handle mechanism.

20. A method for reducing residuals in an eyedrop administrative device comprising the steps of:
applying positive pressure to accelerate an eyedrop out of the device after the eyedrop has left the device; and
utilizing a motion activating mechanism to continue a flow of air exiting the device.
